(19) United States
(12) Patent Application Publication
(10) Pub. No.: US 2007/0010879 A1
(43) Pub. Date: Jan. 11, 2007

(54) Injector system for a lens

(76) Inventors: Gregg Feinerman, Newport Beach, CA (US); Renato Cruz, Rowland Heights, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 11/177,518

(22) Filed: Jul. 7, 2005

(51) Int. Cl. A61F 2/16

(52) U.S. Cl. 623/6.12

(57) ABSTRACT

An intraocular lens delivery system has leading and trailing haptic guides with upper and lower fingers sized and dimensioned to juxtapose at least 10% of at least one of the upper and lower surfaces of the corresponding haptic. The fingers protect the haptics from undesirable folding during preparation of the lens for delivery into an eye of a patient.

### FIELD OF THE INVENTION

The field of the invention is ophthalmology.

### BACKGROUND

An intraocular lens has an optic part for correcting ametropia and a haptic part that supports the optic part when implanted in the eye. Known methods of inserting lenses include using a flexible lens that may be folded or deformed, and then inserting it through a sclerocorneal incision in the eye.

Bosch and Lomb,™ for example, sells a popular lens injector that uses a pusher to feed the lens into a loading chamber. Among other things, the pusher folds the lens within the loading chamber, preparing it for delivery. Unfortunately, lenses inserted using that device often fold up in a manner that hinders the delivery procedure. Still further, the known lens injectors are unsatisfactory for inserting the new Crystalens™ type of lenses because the more complicated haptics in such lenses tend to fold up improperly during delivery.

Thus, there is still a need for a lens injector system that reduces incidences of improper folding of the lens, and that is workable with Crystalens™ type of lenses.

### SUMMARY OF THE INVENTION

The present invention provides apparatus, systems and methods in which improved leading and trailing haptic guides are used to fold of an intraocular lens. The guides are designed to juxtapose at least 10% of at least one of the upper and lower surfaces of the haptics. In preferred embodiments the guides juxtapose at least 20% of the upper and lower surfaces of the leading haptic, more preferably at least 50%, most preferably at least 80%.

Contemplated systems include both the guides and a funnel or other apparatus that folds the lens. Such structures can advantageously be sufficiently transparent so that a user can visualize the intraocular lens as it travels through the apparatus. Such systems are preferably packaged for disposable or single use.

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWINGS

FIG. 1 is a top view of intraocular folding funnel, in which leading and trailing haptic guides are disposed about the haptics of an intraocular lens.

FIG. 2 is a side view of the leading and trailing haptic guides, and the intraocular lens of FIG. 1.

FIG. 3 is a side view of an alternative intraocular folding apparatus, in which leading and trailing haptic guides are disposed about the haptics of an intraocular lens.

FIG. 4A is a plan view of a preferred packaging for an intraocular folding system.

FIG. 4B is a plan view of an alterative packaging for an intraocular folding system.

### DETAILED DESCRIPTION

The present invention relates to devices and methods for preparing and injecting intraocular lenses (IOLs) into an eye, including especially novel leading and trailing guides.

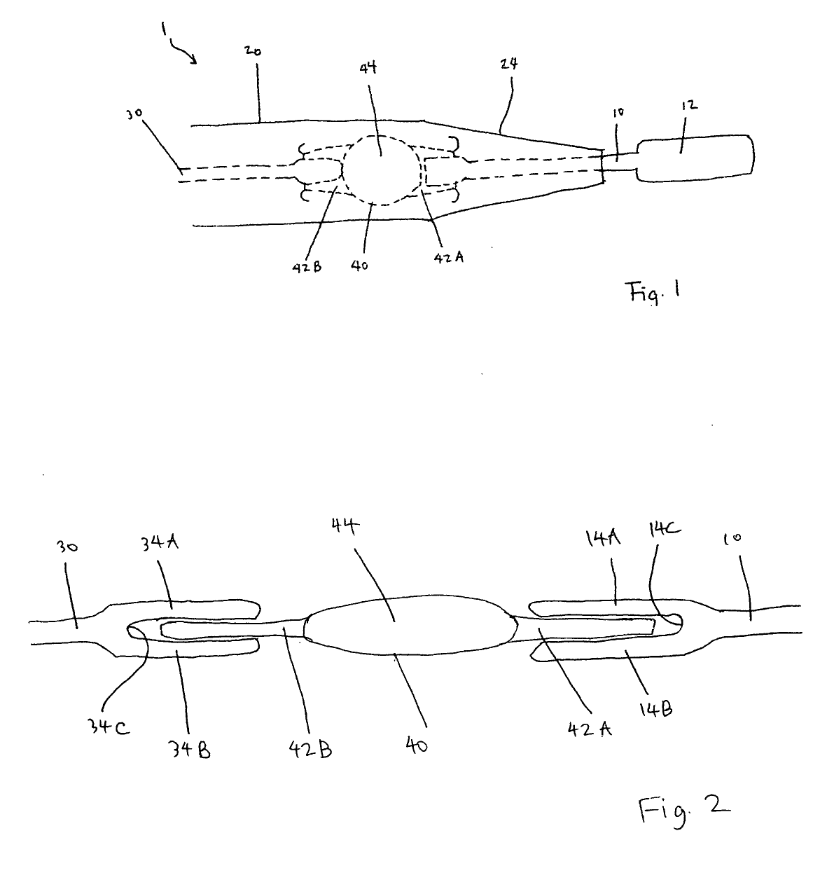

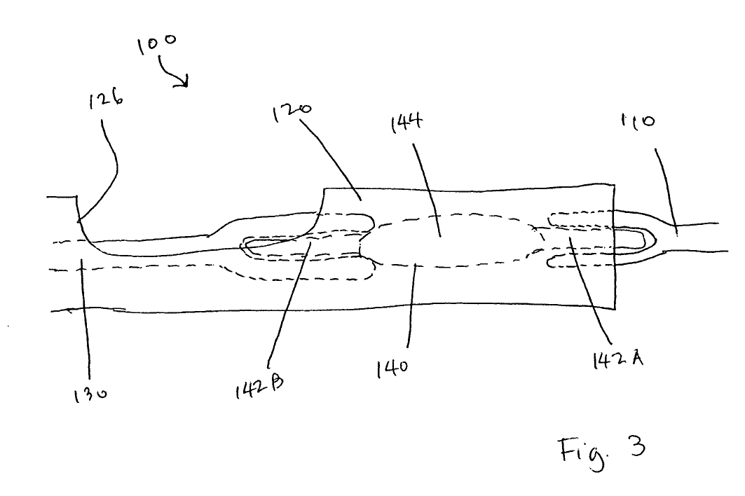

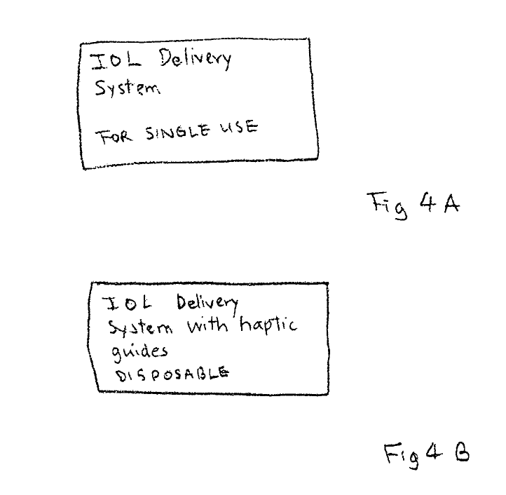

In FIGS. 1-2 an injector system 1 generally includes a leading guide 10, an injector 20, and a trailing guide 30, all of which cooperate to prepare a lens 40 for insertion into a recipient's eye. FIG. 1 depicts the injector system with lens 40 prior to being folded by a funnel 24. The injector 20 is not shown in FIG. 2.

Operation of the system is straightforward. A user first loads a lens 40 into the injector 20. Trailing guide 30 then engages lens 40 and pushes the lens into the funnel 24, as the leading guide 10 leads the lens. When the lens 40 successfully reaches the distal tip of the injector, the leading guide is pulled away from the lens and is disposed of. At this point, the user positions the tip of the injector at an incision in the patient's eye, and injects the lens.

Leading guide 10 generally has a handle 12, holding fingers 14A, 14B that extend to the sides of the leading haptic 42A of the lens, and an intervening web 14C. The fingers 14A, 14B maintain a substantially flat disposition of the lens as it moves through the injector.

In a preferred embodiment, holding fingers 14A and 14B are configured such that these fingers extend across most or all of the leading haptic, and possibly even abuts the leading end of the optic portion 44 of the lens 10. The fingers preferably overlap both the upper and under surfaces of the haptic by at least 10% of the haptic surfaces, more preferably 50%, and most preferably 80%. In some embodiments the fingers may extend all the way from the intervening web 14C to the leading edge of the optic portion of the lens. The larger fingers are generally considered to be more advantageous because they would tend to be better at preventing undesirable folding of the haptic. Of course, it is possible for the upper fingers of the leading guide to have a different shape or size than the lower fingers. There is a tradeoff, however, in that the fingers must be small enough to fit through the narrow end of the funnel.

Trailing guide 30 generally has trailing fingers 34A and 34B, and trailing web 34C. Trailing fingers and trailing web are preferably similar to, indeed may be identical to those of leading guide 10, especially with respect to size and dimension. Similar to leading fingers, the trailing fingers also act to protect the haptics from improper folding during delivery. One difference, however, is that the trailing web 34C, in cooperation with the trailing fingers 34A, 34B, can be used to push the lens through the injector. Thus, the fingers 34A, 34B should be close enough together that they prevent buckling of the haptic as it is being pushed.

All practical sizes, shapes and orientations of leading and trailing fingers are contemplated. Fingers should nevertheless be narrow enough to be inserted into the narrow end 24B of funnel 24, and wide enough to guide lens 40 during delivery. Preferred fingers are about 0.5-2 mm in width, 0.5-1 mm length, and are 0.5-1 mm apart, and more preferably 1 mm wide, 0.75 mm length, and 1 mm apart. Both leading and trailing fingers should be (a) sufficiently flexible to avoid damaging the lens or haptics, and (b) sufficiently rigid to push the lens.

It should be appreciated that the fingers of leading and trailing guides would usually be mirrors of one another. Other contemplated fingers, however, can different in number, configuration and orientation from that shown.

In FIG. 1 injector 20 is funnel shaped. The funnel acts to deform the lens into an appropriately smaller profile for delivery into the eye. It should be noted, however, that passage of the lens 40 through funnel 24 can be performed without leading guide 10. One could, for example, use a top-loading apparatus 120 such as that shown in FIG. 3. Leading guide 110 and trailing guide 120 thereafter engage with the folded lens 140 to ensure smooth passage of the lens 140 within the cylindrical injector 120 during delivery.

Leading guides 10, 110, injectors 20, 120, and trailing guides 30, 130 can each be made of any suitable materials, including especially plastics, Teflon,™ and even metals and composites. Of particular interest are materials that are sufficiently smooth and flexible to avoid damaging the lens and/or haptics, but strong and rigid enough to function as intended. Especially preferred materials are polypropylene or low-density polyethylene. Injectors 20, 120 are preferably made of a material that is sufficiently transparent to allow a user to visualize the lens as it passes through the injectors, and during delivery into a patient's eye.

Lenses 40, 140 can be of any type, including for example, Crystalens™ lenses that have especially large haptics. Nevertheless, other embodiments are contemplated for use with lenses that have no leading or tailing haptic, or only one of them.

It should be apparent to those skilled in the art that many more modifications besides those already described are possible without departing from the inventive concepts herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps could be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A leading haptic guide for an intraocular lens having a leading and a trailing haptic, the leading haptic guide having upper and lower portions sized and dimensioned to fit within an opening of a folding apparatus, and to juxtapose at least 10% of at least one of the upper and lower surfaces of the leading haptic, respectively.

2. The guide of claim 1 wherein the upper and lower portions are sized and dimensioned to juxtapose at least 20% of at least one of the upper and lower surfaces of the leading haptic.

3. The guide of claim 1 wherein the upper and lower portions are sized and dimensioned to juxtapose at least 50% of at least one of the upper and lower surfaces of the leading haptic.

4. The guide of claim 1 wherein the upper and lower portions are sized and dimensioned to juxtapose at least 20% of both of the upper and lower surfaces of the leading haptic.

5. The guide of claim 1 wherein the upper and lower portions are sized and dimensioned to juxtapose at least 50% of both of the upper and lower surfaces of the leading haptic.

6. The guide of claim 1 wherein the upper and lower portions are sized and dimensioned to juxtapose at least 80% of both of the upper and lower surfaces of the leading haptic.

7. The guide of claim 1 further comprising a funnel that operates as the folding apparatus.

8. The guide of claim 1 wherein the folding apparatus is sufficiently transparent such that a user can visualize the intraocular lens through the folding apparatus.

9. An intraocular lens delivery system comprising the leading haptic guide of claim 1, and further comprises a trailing haptic guide having upper and lower portions sized and dimensioned to juxtapose at least 10% of at least one of the upper and lower surfaces of the trailing haptic, respectively.

10. The system of claim 9 wherein the upper and lower portions of the trailing haptic guide are sized and dimensioned to juxtapose at least 20% of at least one of the upper and lower surfaces of the trailing haptic.

11. The system of claim 9 wherein the upper and lower portions of the trailing haptic guide are sized and dimensioned to juxtapose at least 50% of both the upper and lower surfaces of the trailing haptic.

12. The system of claim 9 wherein the leading haptic guide of claim 1 is disposed in a package that designates the system as being disposable.

13. The system of claim 9 wherein the leading haptic guide of claim 1 is disposed in a package that designates the system as being for single use.

14. The system of claim 9 wherein the leading haptic guide comprises a plastic.

15. A trailing haptic guide for an intraocular lens having an optic, a leading and a trailing haptic, the trailing haptic guide having upper and lower portions sized and dimensioned to fit within an opening of a folding apparatus, and to juxtapose at least 10% of at least one of the upper and lower surfaces of the leading haptic, respectively.

16. The guide of claim 15 wherein the guide having a distal portion sized and dimensioned to abut an edge of the optic.
